(19) United States
(12) Patent Application Publication
(10) Pub. No.: US 2024/0390547 A1
(43) Pub. Date: Nov. 28, 2024

(54) DECONTAMINATION SYSTEM FOR A BREATHING APPARATUS

(71) Applicant: DG Devices, LLC, Dayton, OH (US)

(72) Inventors: David Louis Kirschman, Dayton, OH (US); Gregory Carroll, Springboro, OH (US)

(73) Assignee: DG Devices, LLC, Dayton, OH (US)

(21) Appl. No.: 18/668,574

(22) Filed: May 20, 2024

Related U.S. Application Data
(60) Provisional application No. 63/503,751, filed on May 23, 2023.
(60) Provisional application No. 63/579,063, filed on Aug. 28, 2023.

(51) Int. Cl. A61L 9/20; A61M 16/08; A61M 25/00

(52) U.S. Cl. CPC A61L 9/20; A61M 16/08; A61L 2209/11; A61L 2209/12; A61M 2025/0039

(57) ABSTRACT

A breathing apparatus disinfection device and system that has a replaceable inner tube member and helical radiation source for disinfecting air from a ventilator before it reaches a patient. The breathing apparatus disinfection device and system comprises an inner tube member surrounded by a helical shaped radiation source. An outer tube member comprises end caps or modular shield blockers that prevent any radiation from reaching outside the breathing apparatus disinfection device and into the immediate environment, thereby reducing or eliminating unwanted patient or user contamination exposure.

### CROSS-REFERENCE TO RELATED APPLICATIONS

The present application claims priority to provisional U.S. Application Ser. No. 63/503,751, filed May 23, 2023, and 63/579,063, filed Aug. 28, 2023, to which Applicant claims the benefit of the earliest filing date of these applications, which are incorporated herein by reference and made a part hereof.

### BACKGROUND OF THE INVENTION

### 1. Field of the Invention

This application relates to a medical device and, more particularly, to a breathing apparatus having a decontamination system adapted to reduce the risk of ventilator-associated pneumonia (VAP) and also to reduce the risk of life-threatening infection in critically ill patients who are on mechanical ventilation.

### 2. Description of the Related Art

Mechanical ventilation is a life-saving intervention for critically ill patients who experience respiratory failure. Despite its therapeutic importance, the use of mechanical ventilation can also pose significant health risks. One of the most severe complications is ventilator-associated pneumonia (VAP), which increases mortality rates, medical costs, and the duration of hospitalization. Typical measures to minimize the risk of ventilator-associated pneumonia (VAP) include frequent cleaning of the ventilator tubing, but these methods are not foolproof and may lead to biofilm formation and bacterial colonization. Ventilator-associated pneumonia (VAP) is a leading cause of mortality and morbidity in hospitalized patients, particularly those in intensive care units. According to the Center for Disease Control, about 10-20% of patients on ventilators may develop ventilator-associated pneumonia (VAP). Moreover, ventilator-associated pneumonia (VAP) significantly increases hospital costs, with each case adding potentially tens of thousands of dollars to costs on hospital or medical bills.

Ventilator-associated pneumonia (VAP) is a pervasive and life-threatening infection that can occur in critically ill patients who are on mechanical ventilation. As mentioned, ventilator-associated pneumonia (VAP) increases the cost of healthcare and prolongs hospital stays.

Current methods of preventing or reducing ventilator-associated pneumonia (VAP) primarily focus on antibiotic prophylaxis and hygiene measures, which can be insufficient, particularly when drug-resistant pathogens are involved. Therefore, a need exists for an innovative device that can effectively reduce bioburden in the breathing apparatuses used for mechanical ventilation.

### SUMMARY OF THE INVENTION

A disinfection system and device having a continuous disinfection feature of this device addresses a critical gap in the management of patients on ventilators. By preventing the reflux of contaminated secretions, it reduces the chances of introducing pathogens into the patient's airway, thereby decreasing the risk of ventilator-associated pneumonia.

In comparison to current tube-cleaning methods, this invention offers significant advantages. It addresses the limitations of existing methods that primarily rely on manual cleaning or chemical disinfection, both of which may not effectively eliminate all pathogens and could potentially damage the tubes. In contrast, this device leverages non-ionizing radiation for effective, continuous disinfection without the risk of tube damage or chemical exposure.

The modularity of the device is also a novel feature. The design allows for the removal and replacement of the inner tube and the modular shielding means. This allows the device to be re-used by making the inner tube a single use disposable item, and the outer tube, radiation source and housing re-usable.

Furthermore, the housing of the device ensures the containment of radiation and prevents the ingress of fluids. This feature, combined with the safety switch mechanism described herein, further enhances the safety profile of the device, setting it apart from prior solutions. Mechanical ventilation is a life-saving intervention for critically ill patients who experience respiratory failure. Despite its therapeutic importance, the use of mechanical ventilation also poses significant health risks. One of the most severe complications mentioned earlier is ventilator-associated pneumonia (VAP), which increases mortality rates, medical costs, and the duration of hospitalization. Typical measures to minimize the risk of ventilator-associated pneumonia (VAP) include frequent cleaning of the ventilator tubing, but these methods are not foolproof and may lead to biofilm formation and bacterial colonization. The invention fills a crucial gap in the existing healthcare ecosystem by offering a sophisticated yet straightforward solution for reducing bioburden in mechanical ventilation systems. Given the potential for saving lives and reducing healthcare costs, the device is of great significance.

This application introduces a groundbreaking medical device that revolutionizes the disinfection process of ventilator tubing. Through its unique design and use of radiation for continuous disinfection, the device holds the potential to significantly reduce the risk of ventilator-associated pneumonia, thereby enhancing patient safety and reducing healthcare costs.

This application relates to a novel medical device designed to address a pervasive challenge in the field of respiratory care: the risk of ventilator-associated pneumonia (VAP). The invention comprises a decontamination tube system that connects to the tubing of a ventilator system or breathing apparatus. Through the use of non-ionizing radiation, the device disinfects the tube continuously, thereby reducing bioburden and significantly mitigating the risk of ventilator-associated pneumonia (VAP). A distinguishing feature of this device is its ability to prevent reflux of contaminated oronasal secretions into the patient's airway, a shortcoming in many current tube cleaning methods.

The device includes an inner tube, an outer tube, a radiation source, a shielding means, and a housing. The shielding means prevents leakage of radiation, thereby ensuring user and patient safety. The system also includes a separation means, allowing for the inner tube to be removed and replaced by the user while maintaining the outer tube and radiation source.

The present invention proposes a novel device that aims to significantly lower the risk of ventilator-associated pneumonia (VAP) in patients on mechanical ventilation by disinfecting the ventilator tubing through which the air flows. The device utilizes advanced technology to ensure efficient disinfection without interrupting the critical ventilation process.

In one aspect, one embodiment of the invention comprises a decontamination tube system for use within a breathing apparatus or ventilator system comprising an outer tube, an inner tube; shielding means; a radiation source; and a housing. The decontamination tube system is external to a patient, comprising the inner tube in communication with breathing air in the ventilator system and the outer tube substantially surrounding the inner tube and isolated from the breathing air; the decontamination outer tube in operative relationship with the radiation source; the decontamination tube system further comprising a shielding means to prevent leakage of radiation from tube ends of the decontamination tube system, while allowing airflow to pass through; the decontamination tube system further comprising a separation means allowing the inner tube to be removed from the outer tube and replaced by a user while maintaining the outer tube and the radiation source.

In another aspect, another embodiment of the invention comprises a breathing apparatus disinfection device comprising a radiation source comprising an outer tube member; an air passageway comprising a radiation-transmissible inner tube member contiguous with a mechanical ventilator or breathing apparatus; at least one blocking means located proximate to at least one of the ends of the inner tube member or the outer tube member; the at least one blocking means dimensioned to block radiation egress, while substantially preventing linear motion between the inner tube member and the outer tube member; the inner tube member and the outer tube member being substantially concentric and slidable, such that the inner tube member can be removed from the outer tube member without interrupting the outer tube member; the inner tube member and the outer tube member further defining a substantially airtight junction, such that air and fluids from the inner tube member cannot contact the outer tube member.

This invention, including all embodiments shown and described herein, could be used alone or together and/or in combination with one or more of the features covered by one or more of the following list of features:
The outer tube comprises a non-ionizing radiation generating means such as diode, lamp, laser, vacuum tube, or other means and uses ultraviolet radiation in the 240-280 nm range, but near UV, visible, infrared, electron beam, X-ray, and microwave are other means.
The inner tube and/or the outer tube comprise a radiation transmissible means, such as fused quartz, fluorinated ethylene polypropylene, fenestrated material, or other means.
The inner tube is in cooperation with the shielding means, the shielding means comprising a contiguous air passage with internal baffles, angles, fins, spheres, disks, protuberances, wings, foils, or other means to allow air to pass through without significant pressure change, while substantially blocking the passage of radiation.
The shielding means are modular and removal from the tube system and a safety switch may be incorporated to deactivate the radiation source in the absence of the shielding means.
The shielding means further comprises a sealing means to prevent ingress of liquids and or visible radiation between an external environment and the decontamination tube system.
The shielding means comprises at least one internal sphere or spheroid positioned substantially centrally in an air pathway and allowing airflow to pass peripherally, but not substantially allowing visible rays to pass through distal ends of the decontamination tube system, the shielding means comprising a substantially radiation non-transmissible material.
The shielding means comprise an angulated tube of at least 10° and allowing airflow to pass peripherally, but not substantially allowing visible rays to pass through distal ends of the decontamination tube system, the shielding means comprising a substantially radiation non-transmissible material.
The inner tube comprises at least one end mount located at open tube ends for mounting the shielding means or the breathing apparatus, the at least one end mount allowing for application of an extruded quartz tube body or other materials not compatible with mount integration.
The inner tube can be linearly displaced and removed from the outer tube, the outer tube being substantially contiguous along its circumference allowing for uninterrupted circumferential or helical wrapping of the radiation source about the outer tube.
The outer tube is in one or more sections and discontinuous along its circumference, the one or more sections comprising an electrical connection means to allow for powering of multiple radiation sources in a substantially circumferential array.
The safety switch is actuated by the assembly of the inner tubes and the outer tubes, preventing inadvertent activation of the radiation source if the inner tube is not situated within the outer tube and/or if the shielding means are not in place.
The inner tube, the outer tube and the radiation source are contained in a housing dimensioned to substantially enclose the inner tubes, the outer tubes and the radiation source to prevent leakage of visible radiation and ingress of fluids, the housing further comprising a lead means for transmission of an electrical circuit, power supply, control system, and/or switching means.
The outer tube can be substantially integral to the housing, or comprise a discrete tube mounted within the housing.
The shielding modules and/or the inner tube and/or the outer tube, and/or the housing comprise tapered male or female ends for integration into standard breathing apparatus connections, with taper ratios such as 1:40 or 1:36.
The radiation source comprises ultraviolet germicidal radiation, near UV, far UV, infrared, microwave, or any other electromagnetic radiation capable of inactivating microbial species, including bacteria, viruses or spores.
The radiation source comprises an array of radiation-emitting light emitting diodes.
The outer tube member comprises a light-emitting diode array arranged in a substantially helical pattern about a central axis; the central axis defining a centerline of the inner tube member and the outer tube member; the light-emitting diode array associated with a power supply, a controller and a switching means.
The outer tube member comprises a polymer housing incorporating a thermal insulation means.
The inner tube member comprises a substantially radiation transparent wall, such as quartz or fluorinated ethylene polypropylene.
The inner tube member comprises at least one end cap connector; the at least one end cap connector dimensioned to receive the blocking means.
The inner tube member and/or the outer tube member cooperate with at least one blocking means, the at least one blocking means comprising one or more of a blocking flange, a sealing means to the inner tube member or the end cap connector, a sealing means to the outer tube member, a tapered connector for connecting to standard ventilator tubing, an internal angle of at least 30 degrees, or a radiation baffle means.
The blocking means is removable or translatable, allowing parallel sliding of the inner tube member and outer tube member, the inner tube member being completely removable from the outer tube member for cleaning or replacement.

These and other objects and advantages of the invention will be apparent from the following description, the accompanying drawings and the appended claims.

### BRIEF DESCRIPTION OF THE ACCOMPANYING DRAWINGS

FIG. 1 is a view of a breathing apparatus and decontamination device;

FIG. 2A is a view of the assembled breathing apparatus and decontamination device;

FIG. 2B is a sectional view of the breathing apparatus and decontamination device shown in FIG. 2A;

FIG. 3 is another sectional view of the breathing apparatus and decontamination device showing a removeable and replaceable inner tube before it is slidably received in the breathing apparatus and decontamination device;

FIG. 4 is a view showing various details of components of the breathing apparatus and decontamination device, but without the outer housing;

FIG. 5 is a cross-sectional view showing sections of an inner tube and outer tube and a circumferential radiation source that projects radiation inwardly in the inner tube member toward the tubular opening;

FIG. 6 is a view of a modular shielding blocker used in the embodiments of FIGS. 1-8 illustrating the air spacing surrounding a spheroid allowing airflow past the spheroid into or out of the breathing apparatus and decontamination device;

FIG. 7 is another view of the breathing apparatus and decontamination device showing details of a shielding connector safety switch and an inner tube member safety switch;

FIG. 8 is an assembled view of the breathing apparatus and decontamination device showing various details of the angled connectors that prevent leakage of visible radiation from the breathing apparatus and decontamination device;

FIGS. 9A and 9B illustrate inner tube connector embodiments and the use of angled connectors that prevent leakage of visible radiation. The embodiment of FIG. 9A shows a non-integral connector with a tapered connection and FIG. 9B illustrates an integral connector with a tapered connection;

FIG. 10 is a view of another embodiment of the breathing apparatus and decontamination device;

FIG. 11 is a view of a breathing apparatus and decontamination device according to another embodiment where a portion of the housing and the outer tube have been removed to illustrate and show the exposed helical LED array that is housed inside the breathing apparatus and decontamination device and surrounding an inner tube which supports the helical LED array;

FIG. 12 is an exploded view of the embodiments shown in FIGS. 10 and 11; and

FIGS. 13A-13D illustrate the assembly of the inner tube into the breathing apparatus and decontamination device and the use of the blockers on either end which illustrates the process of assembling the breathing apparatus and decontamination device.

### DETAILED DESCRIPTION OF THE PREFERRED EMBODIMENTS

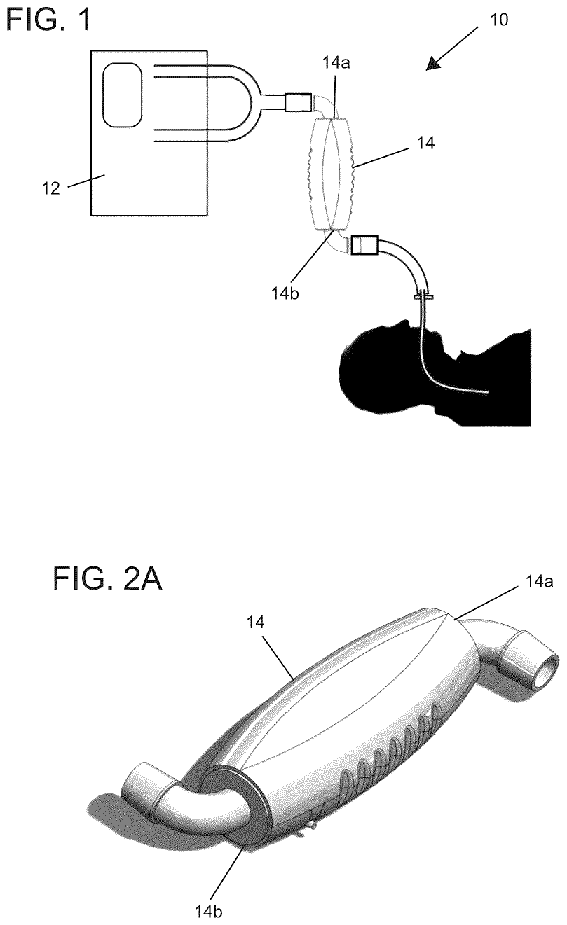

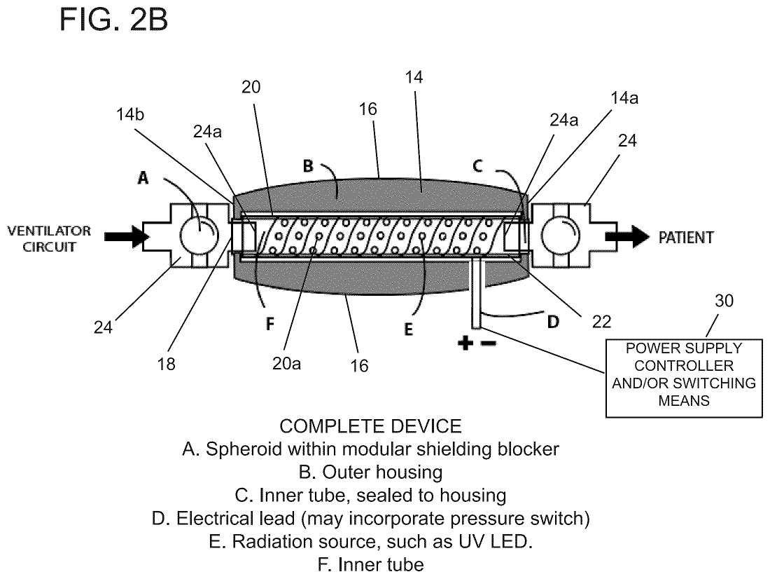

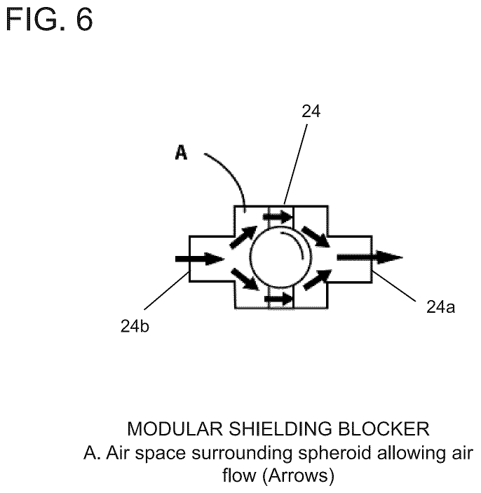

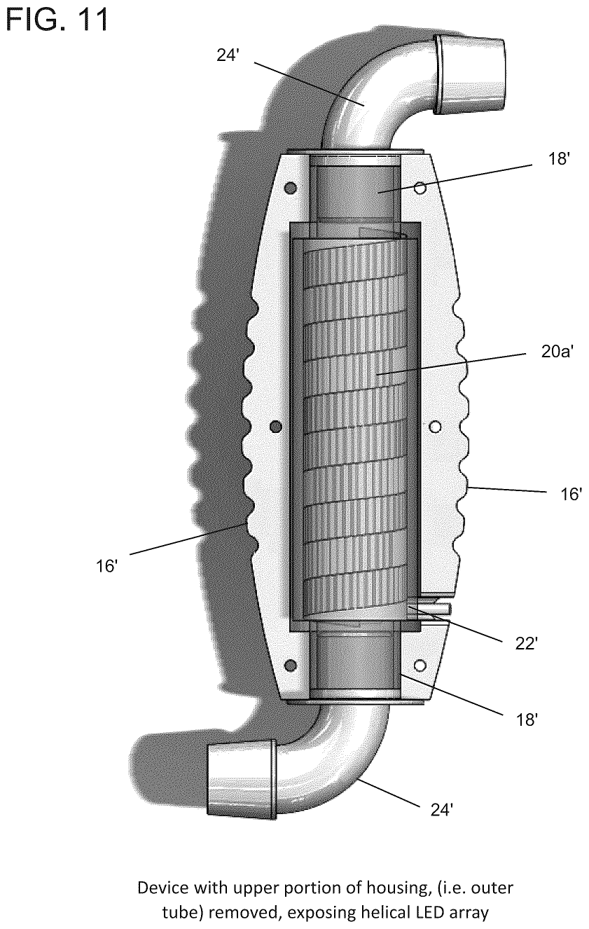

Referring now to FIGS. 1-13D, a breathing apparatus and decontamination device and system 10 are shown. The breathing apparatus and decontamination device and system 10 comprise a ventilator 12 for generating airflow for ventilating a patient. The breathing apparatus and decontamination device and system 10 further comprises a breathing apparatus disinfection device 14 having an inlet end 14a and an outlet end 14b through which air flows to the patient as illustrated. FIG. 1 illustrates the connection between the conventional ventilator tubing (VT) associated with a patient and the outlet end 14b of the breathing apparatus disinfection device 14 and the inlet end 14a coupled to the ventilator 12 so that the breathing apparatus and decontamination device and system 10 are situated between the ventilator 12 and the patient. FIG. 2A shows the breathing apparatus disinfection device 14 fully assembled and before it is inserted between the patient and the ventilator 12. Turning now to FIG. 2B, various details of the breathing apparatus disinfection device 14 will now be described.

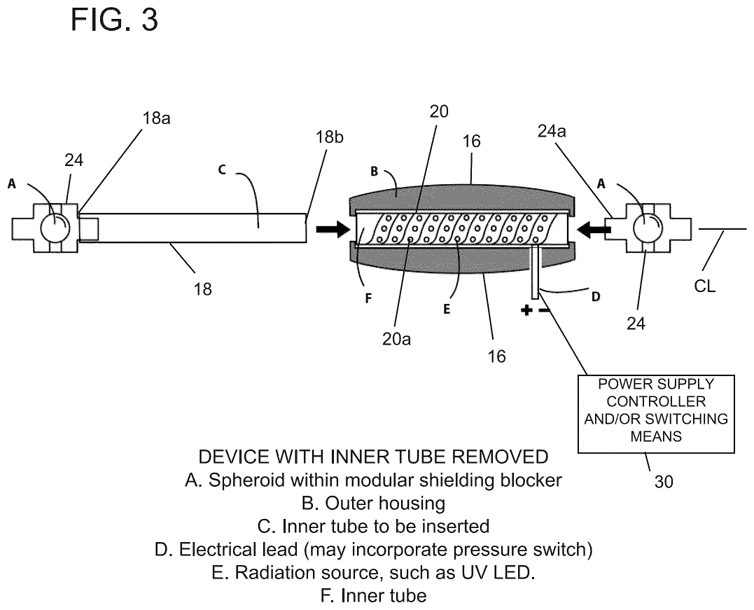

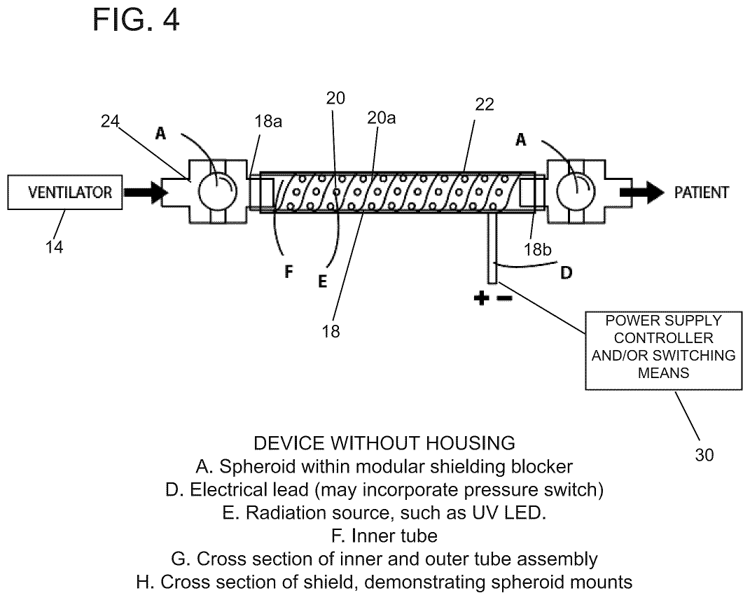

The breathing apparatus disinfection device 14 comprises a housing 16 which in the illustration being described is a two-piece housing and that is adapted to receive an inner tube member 18 which is sealed to the housing 16. As best illustrated in FIGS. 3 and 4, the breathing apparatus disinfection device 14 also comprises a radiation source 20 which in the illustration being described is ultraviolet germicidal radiation, near UV, far UV, infrared, microwave, or any other electromagnetic radiation capable of inactivating microbial species, including bacteria, viruses or spores and/or an array of radiation-emitting diodes 20a, such as a circular array, a tubular array or a helical array. Notice in FIGS. 2B-4 that the radiation source 20 may comprise the array of radiation-emitting diodes 20a. Notice in FIGS. 3 and 4 that the array of radiation-emitting diodes 20a or radiation source 20 is arranged in either a circular pattern or in a substantially helical pattern about a central axis which defines a center line CL (FIG. 3) of both the inner tube member 18 and an outer tube member 22. In the illustration being described, the inner tube member 18 comprises a substantially radiation transparent wall, such as a quartz or fluorinated ethylene polypropylene, that permits the radiation from the radiation source 20 to pass therethrough. The inner tube member 18 comprises at least one end cap connector or spheroid shielding blocker 24 that is adapted to permit air to flow through the breathing apparatus disinfection device 14 while substantially blocking any radiation from exiting the breathing apparatus disinfection device 14 for the safety of the patient and users.

The inner tube member 18 and outer tube member 22 cooperate with the at least one end cap connector or spheroid shielding blocker 24 that comprises one or more blocking flanges, sealing means, gaskets, seals or the like for sealing it inside the inner tube member 18 or an end cap connector.

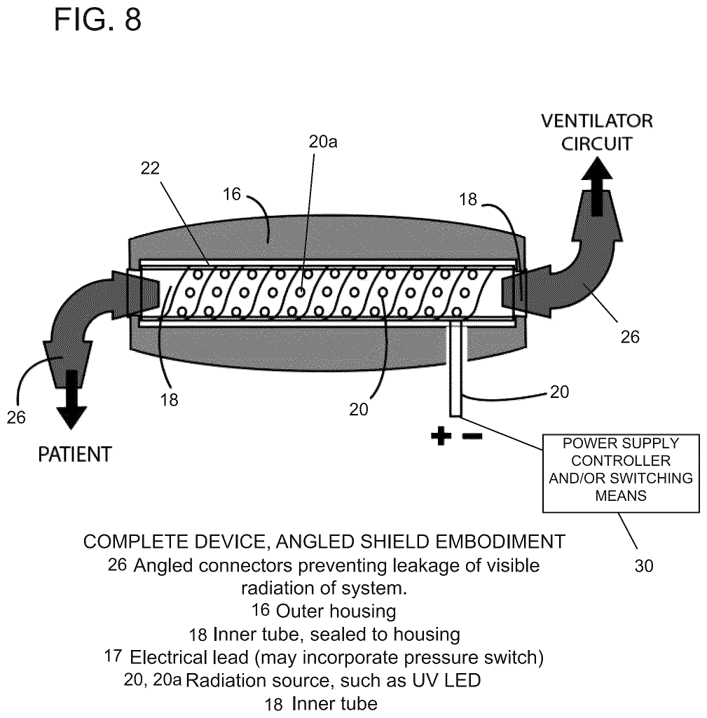

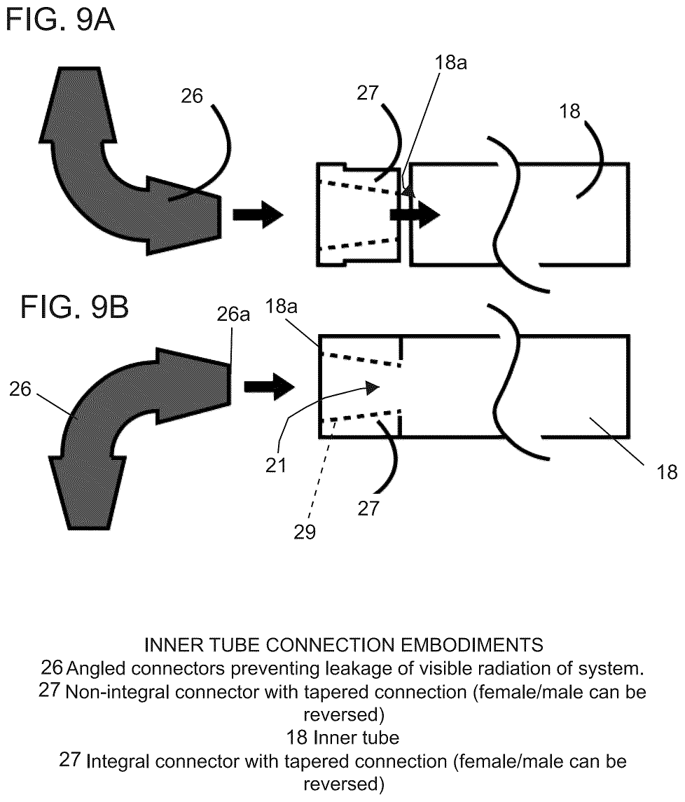

As illustrated in FIGS. 8-9B, a tapered elbow connector 26 may be provided for connecting to standard ventilation tubing in a hospital or medical facility ventilation system. The tapered elbow connector 26 is opaque and does not permit radiation to escape therefrom during use. The power supply 30 is connected to the device via an electrical lead 17 (FIG. 8) that may also incorporate a pressure switch (not shown). In the embodiment of FIG. 9A, the tapered elbow connector 26 is used with a connector 27 that is non-integral which in turn is inserted into the end 18a of the inner tube member 18. FIG. 9B shows another embodiment wherein the connector 27 is integrally formed in an end 18a of the inner tube member 18. The inner tube member 18 has a frusto-conical aperture 21 defined by connector wall 29 that is sized and adapted to receive a male end 26a of the tapered elbow connector 26 as illustrated. Although not shown, the female/male design and configuration could be reversed. The tapered elbow connector 26 is opaque and dimensioned to prevent any radiation from inside the breathing apparatus disinfection device 14 from escaping the inner tube member 18. In the embodiment of FIGS. 1-7, note that the at least one end cap connector or spheroid shielding blocker 24 does not have any frusto-conical apertures, but rather, utilizes the at least one end cap connector or spheroid shielding blocker 24 for preventing radiation leakage. In the illustration being described, the at least one end cap connector or spheroid shielding blocker 24 may be analogous to a check valve that permits air flow through the breathing apparatus disinfection device 14 and ultimately to the patient after the airflow has been irradiated with UV light, for example, and treated as described herein. It also prevents ingress of fluids, such as reflux from the patient. In the illustration being described, the ends 24a, 24b of the at least one end cap connector or spheroid shielding blocker 24 are inserted into ends 18a, 18b of the inner tube member 18 and either press-fit and/or sealed with an appropriate gasket or O-ring (not shown). It should be understood that in the illustration being described, the inner tube member 18, once it has received the spheroid shielding blockers, is sealed and does not permit air or radiation to leak therefrom.

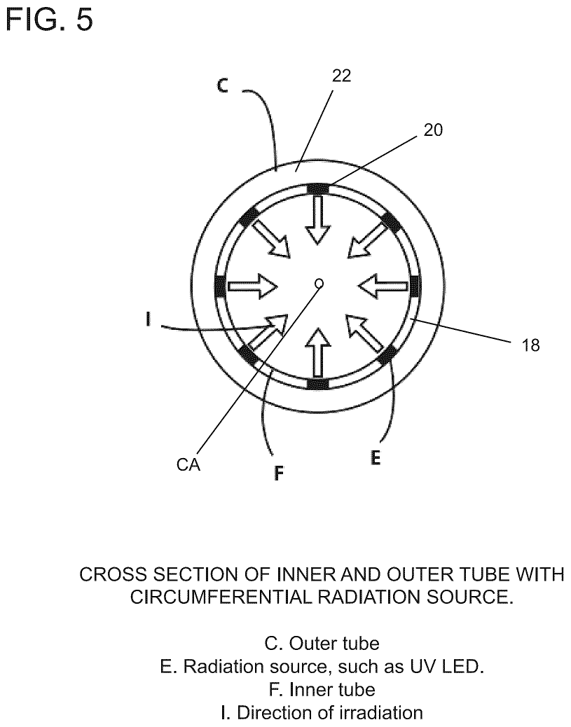

Referring to FIG. 5, the cross-sectional view illustrates the outer tube member 22, the inner tube member 18 and the LED array or radiation source 20 that projects radiation and UV light inward toward the axis CA of the inner tube member 18. This ensures that the air passing through the inner tube member 18 becomes decontaminated and irradiated to reduce or eliminate any bioburden or contamination in the airstream.

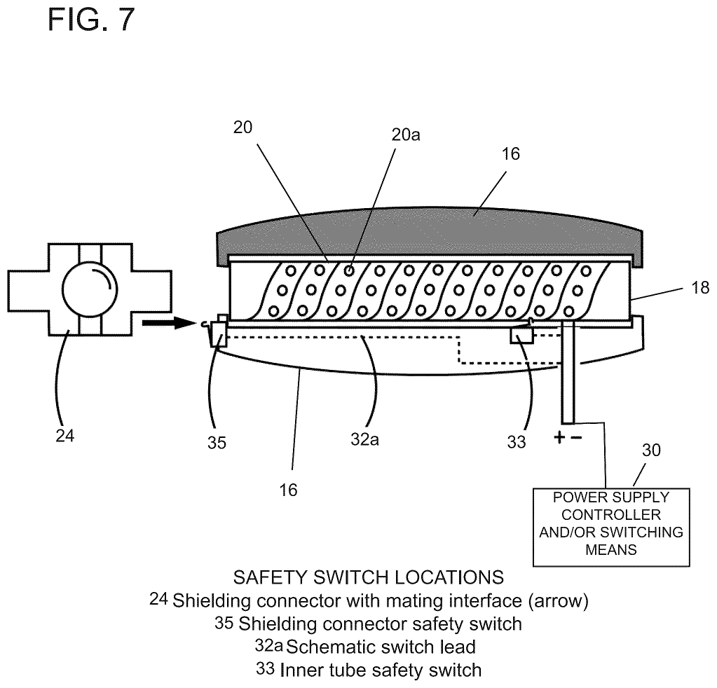

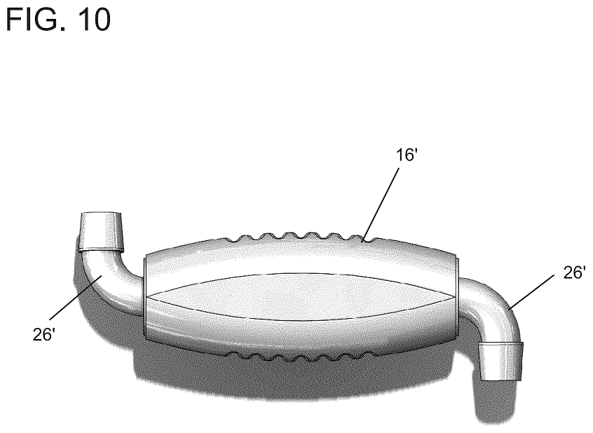

During use, the breathing apparatus disinfection device 14 is assembled into a final form as illustrated in FIGS. 2A and 10 and it is placed in the airstream between the ventilator 12 and the patient as illustrated in FIG. 1. The breathing apparatus disinfection device 14 and radiation source 20 are turned on by a user and powered by a power supply 30 (FIG. 7).

Referring to FIG. 5, a cross-section of the inner tube member 18, the outer tube member 22 and the circumferential radiation source 20 are shown. Notice in FIG. 4 that the breathing apparatus disinfection device 14 is shown without the housing 16 for ease of illustration. Notice how the at least one end cap connector or spheroid shielding blockers 24 are inserted into the ends 18a and 18b of the inner tube member 18 and sealed thereto by conventional means such as a press-fit, seal, gaskets, O-rings or the like.

One significant feature of the illustrations being described is shown in FIG. 3 which illustrates the inner tube member 18 and at least one end cap connector or spheroid shielding blocker 24 being removed from the outer tube member 22 and from the radiation source 20. This feature is significant in that it permits the user to replace the inner tube member 18 after one or a plurality of uses because an inner wall 18c of the inner tube member 18 may become contaminated with bioburden or other contamination. It is contemplated that the inner tube member 18 may be replaceable by the user and reinserted into the breathing apparatus disinfection device 14 as illustrated. In a preferred embodiment, the inner tube member 18 may be replaced after each patient or after a predetermined period of time or other event. It is also contemplated that the inner wall 18c of the inner tube member 18 may optionally comprise at least one or a plurality of airflow interrupters in the form of baffles, ridges, projections or the like (not shown) in order to disturb the airflow so that a dwell time of the airflow as it passes past the radiation source 20 is increased thereby improving the efficiency of the disinfection.

After the inner tube member 18 is inserted inside the outer tube member 22 and the at least one end cap connector or spheroid shielding blocker 24 are inserted into and sealed in the ends 18a and 18b of the inner tube member 18, it becomes sealed inside the housing 16. During use, the operator actuates the power supply 30 which causes an airflow from the ventilator 12 to pass through the breathing apparatus disinfection device 14 and energize the radiation source 20 to decontaminate the airstream which is delivered to the patient after the airflow has been decontaminated.

Referring now to FIGS. 10-13D, in this embodiment, the same or similar parts are identified with the same part numbers, except that a prime mark (“′”) has been added to the part numbers for this different embodiment.

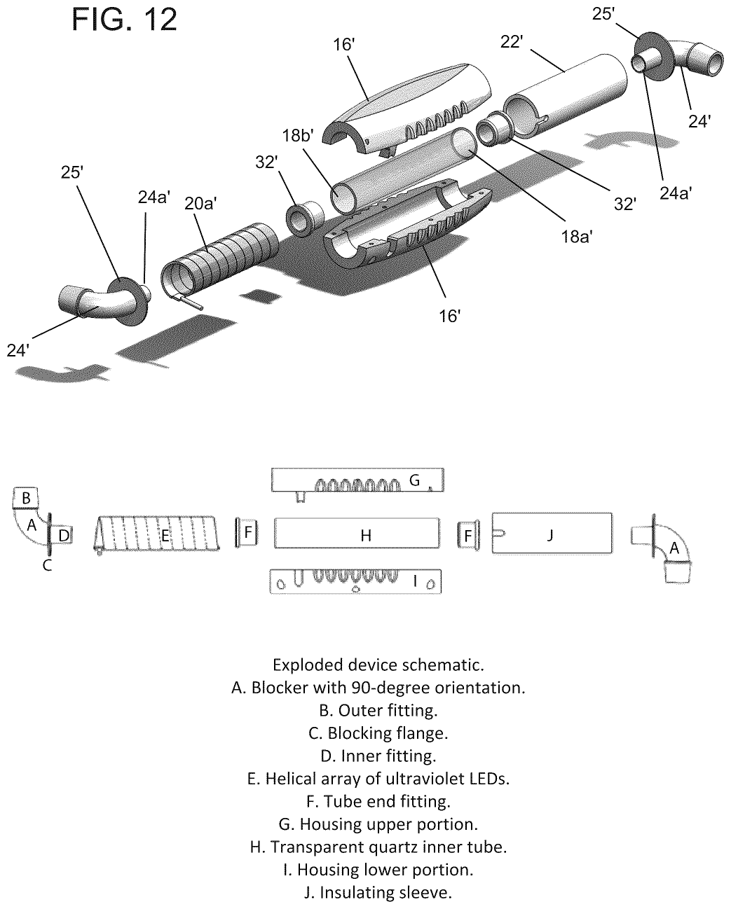

FIG. 12 is an exploded view of this embodiment in perspective. In this embodiment, a plurality of end caps or tube end fittings 32′ for airtight connection to the inner tube member 18′ are shown and are press-fit into the ends 18a′ and 18b′ as illustrated. The end caps 32′ are dimensioned and sized to receive an end 24a′ of the at least one end cap connector or spheroid shielding blocker 24′ with a press-fit or other sealing connection. In the illustration being described, the inner tube member 18′ is transparent and may be a material such as quartz or fluorinated ethylene polypropylene. In the illustration, the outer tube member 22′ provides an insulating sleeve about the inner tube member 18′ as illustrated. As with the prior embodiment, the inner tube member 18′ is replaceable by the user and preferably is replaced after each use or after a predetermined period of time to reduce or eliminate the risk of patient infection or ventilator-associated pneumonia (VAP). Note that each of the at least one end cap connector or spheroid shielding blocker 24′ has a blocking or sealing flange 25′ that prevents lateral movement of the inner tube member 18′ with respect to the outer tube member 22′ and also facilitates sealing the inner tube member 18′ to the housing 16′. The blocking or sealing flange 25′ may be integrally formed in the at least one end cap connector or spheroid shielding blocker 24′. As with the prior embodiment, the at least one end cap connector or spheroid shielding blocker 24′ and the blocking or sealing flange 25′ facilitate preventing radiation leaking from outside of the breathing apparatus disinfection device 14′.

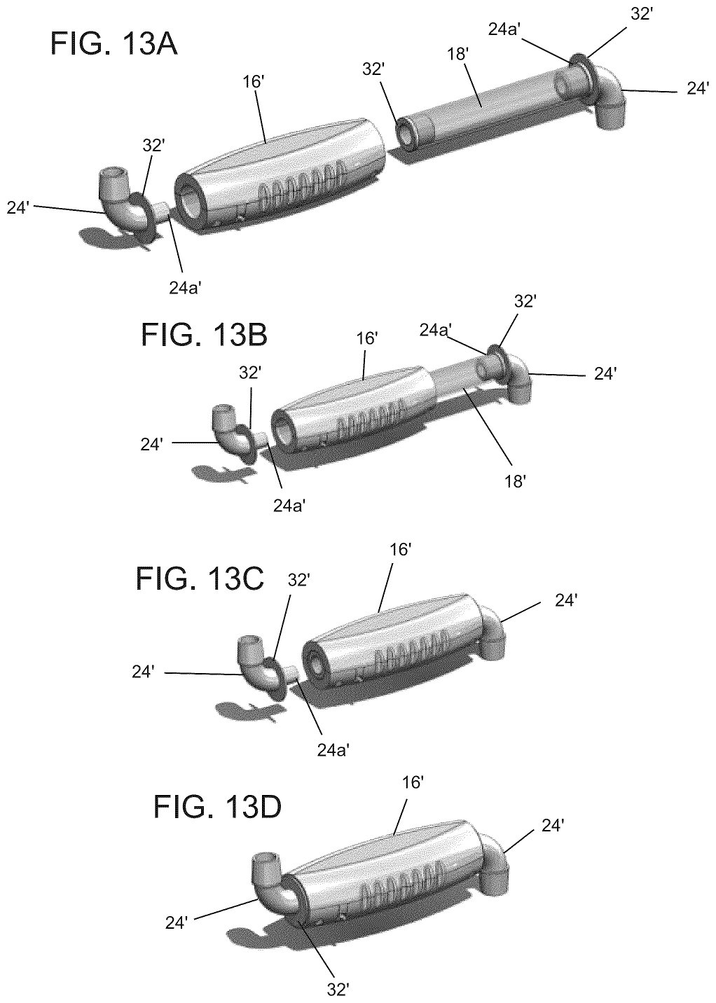

Referring now to FIGS. 13A-13D, as with the embodiment described earlier herein, the inner tube member 18′ is removeable and replaceable and can be separated from the housing 16′. Once the inner tube member 18′ is removed from the housing 16′, the inner tube member 18′ may be changed and a new inner tube member 18′ is inserted into the housing 16′ as illustrated in FIGS. 13A-13D. Once the assembly process is complete, the breathing apparatus disinfection device 14′ can be used with the ventilator 12′ and a patient as described earlier. Advantageously, the breathing apparatus disinfection device 14′ comprises the at least one end cap connector or spheroid shielding blocker 24′ that prevents leakage of radiation, thereby ensuring the safety of the user and the patient. The breathing apparatus disinfection device 14′ also includes means for separating or separation means as described for allowing the inner tube member 18′ to be removed and replaced by the user while maintaining the outer tube member 22′ and radiation source 20′ in the housing 16′.

Advantageously, the breathing apparatus disinfection device 14′ provides continuous disinfection which addresses a critical gap in the management of patients who are on ventilators 12′. By preventing the reflux of contaminated secretions, the chances of introducing pathogens into the patient's airway is reduced and reducing the risk of ventilator-associated pneumonia (VAP). By having the ability to replace the inner tube member 18′, the breathing apparatus disinfection device 14′ can be repeatedly used with new inner tube members 18′, thereby making the breathing apparatus disinfection device 14′ reusable with the same patient or even different patients.

### ADDITIONAL CONSIDERATIONS

The following are some additional considerations or features of the breathing apparatus disinfection device 14.
1. The outer tube member 22 may comprise a non-ionizing radiation generating means, such as diode, lamp, laser, vacuum tube, or other means. The preferred embodiment uses ultraviolet radiation in the 240-280 nm range, but near UV, visible, infrared, electron beam, X-ray, and microwave are other means that can be used as well.
2. The inner tube member 18 and/or outer tube member 22 may comprise a radiation transmissible means, such as fused quartz, fluorinated ethylene polypropylene, fenestrated material, or other means.
3. The inner tube member 18 cooperates with the at least one end cap connector or spheroid shielding blocker 24. The at least one end cap connector or spheroid shielding blocker 24 comprises a contiguous air passage with internal baffles, angles, fins, spheres, disks, protuberances, wings, foils, or other means to allow air to pass through without significant pressure change, while substantially blocking the passage of radiation.
4. The at least one end cap connector or spheroid shielding blocker 24 is modular and removable from the breathing apparatus disinfection device 14. Optionally, a safety switch 35 (FIG. 7) may be incorporated to deactivate radiation source 20 in the absence of the at least one end cap connector or spheroid shielding blocker 24. The safety switch 35 is coupled to the controller 30 by a schematic switch lead 32a and if the at least one end cap connector or spheroid shielding blocker 24 is not fully inserted in the housing 16, the switch 35 is not activated and the radiation source 20 is not energized. Another safety switch 33 for the inner tube member prevents the radiation source 20 from being energized if the inner tube member 18 is not fully seated in the housing 16.
5. The at least one end cap connector or spheroid shielding blocker 24 may further comprise the sealing means 25 to prevent ingress of liquids and or visible radiation between the external environment and the breathing apparatus disinfection device 14.
6. The at least one end cap connector or spheroid shielding blocker 24 may comprise at least one internal sphere or spheroid positioned substantially centrally in the air pathway and allowing airflow to pass peripherally, but not substantially allowing visible rays to pass through distal ends of the breathing apparatus disinfection device 14. The at least one end cap connector or spheroid shielding blocker 24 comprises a substantially radiation non-transmissible material so radiation does not leak.
7. The at least one end cap connector or spheroid shielding blocker 24 may comprise an angulated tube of at least 10° and allows airflow to pass peripherally, but does not substantially allow visible rays to pass through distal ends of the breathing apparatus disinfection device 14. The at least one end cap connector or spheroid shielding blocker 24 comprises a substantially radiation non-transmissible material.
8. The inner tube member 18 comprises at least one end mount located at the open tube ends 18a, 18b for the mounting of the at least one end cap connector or spheroid shielding blocker 24 or the breathing apparatus disinfection device 14. The end mount allows for application of an extruded quartz tube body or other materials not compatible with mount integration.
9. The inner tube member 18 can be linearly displaced and removed from the outer tube member 22. The outer tube member 22 can be substantially contiguous along its circumference. The continuity allows for uninterrupted circumferential or helical wrapping of the linear radiation source 20 about the inside or outside of the outer tube member 22.
10. The outer tube member 22 may be in one or more sections and discontinuous along its circumference. The breathing apparatus disinfection device 4 comprises an electrical connection means for coupling to the power supply 30 to allow for powering of multiple radiation sources 20 in a substantially circumferential array.
11. The breathing apparatus disinfection device 14 may also comprise the safety switches 33, 35 that are actuated by the assembly of the inner tube member 18 and the outer tube member 22, preventing inadvertent activation of the radiation source 20 if the inner tube member 18 is not situated within the outer tube member 22 and/or if the at least one end cap connector or spheroid shielding blocker 24 are not in place.
12. The inner tube member 18, the outer tube member 22 and the radiation source 20 are contained in the housing 16 dimensioned to substantially enclose the inner and outer tube members 18, 22 and the radiation source 20, prevent leakage of visible radiation, and prevent ingress of fluids. The housing 16 may further comprise a lead means for transmission of an electrical circuit, power supply 30, control system, and/or switching means.
14. The outer tube member 22 can be substantially integral to the housing 16 or comprise a discrete tube mounted within the housing 16.
15. The at least one end cap connector or spheroid shielding blocker 24 and/or the inner tube member 18 and/or the outer tube member 22, and/or the housing 16 comprise tapered male or female ends for integration into standard breathing apparatus connections, with taper ratios such as 1:40 or 1:36.
16. The radiation source 20 comprises ultraviolet germicidal radiation, near UV, far UV, infrared, microwave, or any other electromagnetic radiation capable of inactivating microbial species, including bacteria, viruses or spores.
17. The radiation source 20 comprises an array of radiation-emitting light emitting diodes 20a.
18. The outer tube member 22 comprises a light-emitting diode array arranged in a substantially cylindrical or helical pattern about a central axis; the axis defining the centerline of the inner and outer tube members 18, 22; the array associated with power supply, controller and switching means.
19. The outer tube member 22 comprises a polymer housing incorporating a thermal insulation means in the two-piece housing 16.
20. As mentioned earlier, the inner tube member 18 comprises a substantially radiation transparent wall, such as quartz or fluorinated ethylene polypropylene, and the inner tube member 18 comprises at least one end cap connector; the end cap dimensioned to receive the blocking means.
22. The inner and/or the outer tube member 18, 22 cooperate with the at least one end cap connector or spheroid shielding blocker 24 that comprises one or more of a blocking flange 25, a sealing means to the inner tube or the end cap connector, a sealing means to the outer tube member 22, a tapered connector for connecting to standard ventilator tubing, an internal angle of at least 30 degrees, or a radiation baffle means.
23. The at least one end cap connector or spheroid shielding blocker 24 is removable or translatable, allowing parallel and relative movement, sliding or separation of the inner and outer tube members 18, 22, the inner tube member 18 being completely removable from the outer tube member 22 for cleaning or replacement.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

1. A decontamination tube system for use within a breathing apparatus or ventilator system comprising:
an outer tube;
an inner tube;
a shielding means;
a radiation source; and
a housing;
said decontamination tube system being external to a patient, comprising said inner tube in communication with breathing air in the ventilator system and said outer tube substantially surrounding said inner tube and isolated from said breathing air;
said outer tube in operative relationship with said radiation source;
said decontamination tube system further comprising a shielding means to prevent leakage of radiation from tube ends of said decontamination tube system, while allowing airflow to pass through;
said decontamination tube system further comprising a separation means allowing said inner tube to be removed from said outer tube and replaced by a user while maintaining said outer tube and said radiation source.

2. The decontamination tube system of claim 1 wherein said outer tube comprises a non-ionizing radiation generating means such as diode, lamp, laser, vacuum tube, or other means and uses ultraviolet radiation in the 240-280 nm range, but near UV, visible, infrared, electron beam, X-ray, and microwave are other means.

3. The decontamination tube system of claim 1 wherein said inner tube and/or said outer tube comprise a radiation transmissible means, such as fused quartz, fluorinated ethylene polypropylene, fenestrated material, or other means.

4. The decontamination tube system of claim 1 wherein said inner tube is in cooperation with said shielding means, said shielding means comprising a contiguous air passage with internal baffles, angles, fins, spheres, disks, protuberances, wings, foils, or other means to allow air to pass through without significant pressure change, while substantially blocking the passage of radiation.

5. The decontamination tube system of claim 1 wherein said shielding means are modular and removal from said tube system and a safety switch may be incorporated to deactivate said radiation source in the absence of said shielding means.

6. The decontamination tube system of claim 1 wherein said shielding means further comprises a sealing means to prevent ingress of liquids and or visible radiation between an external environment and said decontamination tube system.

7. The decontamination tube system of claim 1 wherein said shielding means comprises at least one internal sphere or spheroid positioned substantially centrally in an air pathway and allowing airflow to pass peripherally, but not substantially allowing visible rays to pass through distal ends of said decontamination tube system, said shielding means comprising a substantially radiation non-transmissible material.

8. The decontamination tube system of claim 1 wherein said shielding means comprise an angulated tube of at least 10° and allowing airflow to pass peripherally, but not substantially allowing visible rays to pass through distal ends of said decontamination tube system, said shielding means comprising a substantially radiation non-transmissible material.

9. The decontamination tube system of claim 1 wherein said inner tube comprises at least one end mount located at open tube ends for mounting said shielding means or said breathing apparatus, said at least one end mount allowing for application of an extruded quartz tube body or other materials not compatible with mount integration.

10. The decontamination tube system of claim 1 wherein said inner tube can be linearly displaced and removed from said outer tube, said outer tube being substantially contiguous along its circumference allowing for uninterrupted circumferential or helical wrapping of said radiation source about said outer tube.

11. The decontamination tube system of claim 1 wherein said outer tube is in one or more sections and discontinuous along its circumference, said one or more sections comprising an electrical connection means to allow for powering of multiple radiation sources in a substantially circumferential array.

12. The decontamination tube system of claim 1 wherein a safety switch is actuated by the assembly of said inner tubes and said outer tubes, preventing inadvertent activation of said radiation source if said inner tube is not situated within said outer tube and/or if said shielding means are not in place.

13. The decontamination tube system of claim 1 wherein said inner tube, said outer tube and said radiation source are contained in a housing dimensioned to substantially enclose said inner tubes, said outer tubes and said radiation source to prevent leakage of visible radiation and ingress of fluids, said housing further comprising a lead means for transmission of an electrical circuit, power supply, control system, and/or switching means.

14. The decontamination tube system of claim 1 wherein said outer tube can be substantially integral to said housing, or comprise a discrete tube mounted within said housing.

15. The decontamination tube system of claim 1 wherein said shielding means and/or said inner tube and/or said outer tube, and/or said housing comprise tapered male or female ends for integration into standard breathing apparatus connections, with taper ratios such as 1:40 or 1:36.

16. A breathing apparatus disinfection device comprising:
a radiation source comprising an outer tube member;
an air passageway comprising a radiation-transmissible inner tube member contiguous with a mechanical ventilator or breathing apparatus;
at least one blocking means located proximate to at least one end of said inner tube member or said outer tube member;
said at least one blocking means dimensioned to block radiation egress, while substantially preventing linear motion between said inner tube member and said outer tube member;
said inner tube member and said outer tube member being substantially concentric and slidable, such that said inner tube member can be removed from said outer tube member without interrupting said outer tube member;
said inner tube member and said outer tube member further defining a substantially airtight junction, such that air and fluids from said inner tube member cannot contact said outer tube member.

17. The ventilator tubing disinfection device of claim 16 wherein said radiation source comprises ultraviolet germicidal radiation, near UV, far UV, infrared, microwave, or any other electromagnetic radiation capable of inactivating microbial species, including bacteria, viruses or spores.

18. The ventilator tubing disinfection device of claim 16 wherein said radiation source comprises an array of radiation-emitting light emitting diodes.

19. The ventilator tubing disinfection device of claim 16 wherein said outer tube member comprises a light-emitting diode array arranged in a substantially helical pattern about a central axis;
said central axis defining a centerline of said inner tube member and said outer tube member;
said light-emitting diode array associated with a power supply, a controller and a switching means.

20. The ventilator tubing disinfection device of claim 16 wherein said outer tube member comprises a polymer housing incorporating a thermal insulation means.

21. The ventilator tubing disinfection device of claim 16 wherein said inner tube member comprises a substantially radiation transparent wall, such as quartz or fluorinated ethylene polypropylene.

22. The ventilator tubing disinfection device of claim 16 wherein said inner tube member comprises at least one end cap connector;
said at least one end cap connector dimensioned to receive said blocking means.

23. The ventilator tubing disinfection device of claim 22 wherein said inner tube member and/or said outer tube member cooperate with at least one blocking means, said at least one blocking means comprising one or more of a blocking flange, a sealing means to said inner tube member or said end cap connector, a sealing means to said outer tube member, a tapered connector for connecting to standard ventilator tubing, an internal angle of at least 30 degrees, or a radiation baffle means.

24. The ventilator tubing disinfection device of claim 16 wherein said blocking means is removable or translatable, allowing parallel sliding of said inner tube member and outer tube member, said inner tube member being completely removable from said outer tube member for cleaning or replacement.
